(19) United States
(12) Patent Application Publication
(10) Pub. No.: US 2006/0122664 A1
(43) Pub. Date: Jun. 8, 2006

(54) Cochlear ear implant

(76) Inventors: Michael Sacha, Chanhassen, MN (US); Jay Chang, Fullerton, CA (US); Seyol David Choye, Fullerton, CA (US)

Correspondence Address: DRUMMOND & DUCKWORTH, Suite 500, 4590 MacArthur Blvd., Newport Beach, CA, 92660, US

(21) Appl. No.: 11/148,736

(22) Filed: Jun. 8, 2005

Related U.S. Application Data
(60) Provisional application No. 60/634,198, filed on Dec. 7, 2004.

(51) Int. Cl. A61N 1/18

(52) U.S. Cl. 607/57

(57) ABSTRACT

A simple cochlear implant is provided that avoids excavation of the mastoid bone. The cochlear implant includes one or more modules for housing the active and passive electronics, and an electrode array for stimulating the nerves of the cochlea. The cochlear implant may include a single modular unit which houses all of the electronics including processor, power supply and microphone. The single modular unit is positioned within the soft tissue behind the ear pinna. Alternatively, the cochlear implant includes two modular units. The first module is implanted within the soft tissue between the ear pinna and mastoid bone. Meanwhile, the second unit is an external module which may be positioned at various external locations. The exterior module may transmit electrical signals to the implanted module through various communication connections including direct electrical contact or through an electromagnetic link. The electrode array is surgically routed from the implanted module in the soft tissue to the cochlea without entering the ear canal by positioning the electrode array between the mastoid bone and the skin of the auditory canal. The electrode array is then routed around the tympanic membrane and through the middle ear to the cochlea. In addition, may be routed along a channel formed in the mastoid bone or through a hole formed in the Spine of Henle for further supporting the electrode array.

### RELATED APPLICATIONS

This application is a continuation-in-part of my co-pending U.S. Provisional Application Ser. No. 60/634,198, filed Dec. 7, 2004.

### BACKGROUND OF THE INVENTION

The present invention relates to a cochlear implant device ideally suited for those humans who are profoundly deaf, where conventional hearing aids are of limited or no value. A profoundly deaf ear is typically one in which the sensory receptors of the inner ear, called hair cells, are damaged or diminished. Unfortunately, the use of a hearing aid does not enable such an ear to process sound. Meanwhile, cochlear implants bypass damaged hair cells and directly stimulate the hearing nerves with an electrical current, allowing individuals who are profoundly or totally deaf to hear.

The ear is an amazing structure consisting of three main parts including the outer ear, the middle ear and the inner ear. The outer ear includes the visible outer portion of the ear called the auricle and the auditory canal. The middle ear includes the eardrum and three tiny bones commonly referred to as the “hammer”, “anvil” and “stirrup”, and medically referred to as the “malleus”, “incus” and “stapes”. The inner ear comprises the fluid filled coil-shaped cochlea which contains thousands of tiny hair cells.

When the ear is functioning normally, sound waves are collected by the outer ear and directed through the ear canal to the middle ear. The sound waves strike the eardrum, also called the tympanic membrane, and cause it to vibrate. This vibration creates a chain reaction in the three tiny bones of the middle ear. Motion of these bones causes movement of the fluid within the cochlea. Meanwhile, the hair cells within the cochlea convert these mechanical vibrations into electrical impulses which are sent to the hearing nerves. Thereafter, the hearing nerves transmit electrical energy to the brain which interprets the energy as “sound”.

Unfortunately, some people suffer damage or depletion of the hair cells resulting in profound hearing loss. In these cases, electrical energy cannot be generated and transmitted to the brain. Without these electrical impulses, the hearing nerves cannot carry messages from the cochlea to the brain and even the loudest of sounds cannot be heard.

Cochlear implants have been developed to enable those persons suffering from profound hearing loss to hear. Although the hair cells in the cochlea may be damaged, there are usually some surviving hearing nerves. A cochlear implant works by bypassing the damaged hair cells and directly stimulating the surviving hearing nerves with an electrical signal. The stimulated hearing nerves then carry the electrical signals to the brain which are interpreted by the brain as sound.

Typically, cochlear implants include two modular units. The first unit is an external module which typically resides behind the ear auricle, in the temporal bone region. It includes external microphones that sense acoustic pressure waves and then converts them to electrical signals. The electrical signals are processed by a signal processor which typically amplifies and converts the electrical signals into stimulation signals. The second module is an implanted unit which is located in a temporal bone excavation typically located just behind the auricle. Typically, the outer module communicates with the implanted module via transcutaneous induction. Across this inductive link, audio information is transmitted as well as energy to power the electronics of the implanted module. Within this implanted module, algorithms are implemented that allow for various methodologies of electrode stimulation. The implanted module connects to an electrode array which extends from the excavated area to the cochlea, where the array end is implanted within the scala tympani duct. This nerve stimulation is then interpreted by the brain as sound.

Unfortunately, cochlear implants suffer from significant drawbacks. The main problem with conventional cochlear implants is that during the implantation phase, residual hearing can be destroyed. Since the length of typical electrode arrays extend beyond the first cochlear bend, it is forced into the curvature by deflecting off the cochlear wall, causing damage to the Stria Vascularus, Spiral Ligament, and even the Basilar Membrane regions. This damage, potentially, precludes these patients from utilizing future technological developments in hearing science.

Another problem is that traditional cochlear implants require temporal bone excavation, within which the implanted electronics module is placed and through which the electrode array is presented to the cochlea. To accomplish this, the cochlear implants must be surgically introduced via a complicated and risky procedure known as the facial recess mastoidectomy. The facial recess mastoidectomy requires the removal of, or drilling through, the mastoid bone to gain access to the cochlea. The conventional surgical approach, removing the mastoid bone to make room for an implant module, can take up to five hours to perform, though three hours is typical, and requires the patient to be placed under general anesthesia. In addition, the operation requires a two-night stay in a hospital, and post operatively, the healing process usually takes about a month. After that month, the patient is introduced to their external module and the implant is finally activated. Numbness in the vicinity of the ear can last up to 6 months after the operation. There are several risk factors associated with typical cochlear implants—risks associated with facial paralysis, loss of taste, dizziness, and ringing in the ear. The operation requires the patient to be placed under general anesthesia which represents an additional risk. In addition, patients that cannot tolerate general anesthesia are excluded from participating in this technology.

Cochlear implants are also very expensive, requiring surgery, anesthesia, a hospital stay, and cochlear programming as each cochlear implant must also be programmed individually for each user which is also expensive and time consuming. The entire procedure is prohibitively expensive and impractical for the vast majority of deaf people in the world. Moreover, few doctors in developing countries have the sophistication, expertise and equipment to perform a facial recess mastoidectomy.

Thus, there is a significant need for a cochlear implant which is inexpensive and involves a minimum of invasive surgery.

### SUMMARY OF THE INVENTION

The present invention addresses the aforementioned disadvantages by providing several improved cochlear implant constructions that do not require drilling through the mastoid bone and do not destroy residual hearing. To accomplish these advantages, the cochlear implant of the present invention includes a module which is surgically implanted in the soft tissue behind the pinna but which does not require drilling through the mastoid bone.

The cochlear implant of the present invention includes one or more modules for housing the active and passive electronics and an electrode array for stimulating the nerves of the cochlea. The cochlear implant may include a single modular unit which houses all of the active electronics including processor, power supply and microphone. The single modular unit is positioned within the soft tissue behind the ear pinna. However, alternatively, the cochlear implant may include two or more modular units. The first module is implanted within the soft tissue between the ear pinna and mastoid bone. Meanwhile, the second unit is an external module which may be positioned at various external locations such as behind-the-ear (BTE) or within the ear canal. For patients that cannot accept an in-the-ear object or accept the behind-the-ear location (BTE), other ear locations can be used. This external module communicates signals to the implanted ear module, which in turn, transmits electrical stimulus signals to the electrode array.

The exterior ear module includes an external microphone that senses acoustic pressure waves and then converts them to electrical signals. The electrical signals are processed by a signal processor which typically provides amplification and conversion of the signals into electrical stimulus signals designed to stimulate nerves within the cochlea.

As opposed to the easily removable exterior ear module, the implanted ear module is surgically implanted in the soft tissue behind the pinna. Preferably, the implanted module is provided in the shape of a tube having an elongate body and having a sufficiently small diameter so that it can be inserted into the soft tissue between the pinna and mastoid bone by a “piercing” operation. The implanted ear module is of simple construction and preferably does not contain active electronics. Instead, it is preferably a simple passive module for relaying signals to the electrode array. The purpose of the implanted ear module is to receive signals, which may be pulsatile or amplitude modulated in nature, from the exterior ear module and convey those signals to the cochlear electrode array.

The exterior module may transmit electrical signals to the implanted module through various communication connections known to those skilled in the art including direct electrical contact. For example, the modules may be electrically connected using miniaturized electrical connectors or through a transcutaneous induction link. If transmitting signals using an electrical connector, the connector should be biocompatible and miniature in construction and provide relative ease of connectability and accessibility for a surgeon. Across this link, audio information is transmitted as well as energy to power any electronics of the implanted module.

Though a direct electrical connection between the exterior module and implanted module may be employed, the communication between the exterior and implanted ear modules is preferably accomplished using a transcutaneous induction link. To this end, the exterior ear module includes a primary induction coil that produces a variable electromagnetic field in response to electrical signals sent from the processor which is, in turn, transmitted to the secondary coil through induction. In a preferred embodiment, the primary induction coil is positioned to be located in close proximity to the interior ear module. Meanwhile, the interior ear module includes a secondary coil for the inductive link. The inductance of the secondary coil integrates this induction signal and passively extracts audio information transmitted by the primary coil to produce stimulus signals.

The cochlear implant further includes an electrode array which extends from the implanted module of either the single module construction or double module construction. The electrode array is then routed by various paths to the cochlea where the array end is implanted within the scala tympani duct. The electrode array is a simple structure including the implanted active electrode, the return electrode, and a biocompatible miniature connector. The electrode array preferably includes an electrode have a single strand of wire having a diameter of 5-10 mil (one thousandth of an inch) coated with an insulator 5-10 microns (one millionth of an inch) thick. Preferably, the electrode wires are insulated and made of particularly soft metal such as substantially pure platinum. Alternatively, preferably the electrode wire includes an uncharacteristically high amount platinum to iridium having a ratio of greater than 90%:10%.

In preferred embodiments, implantation of the electrode array does not require surgical excavation of the mastoid bone to route the electrode array from the implanted module to the cochlea. Instead, the electrode array is positioned to pass within the ear canal and through or underneath the tympanic membrane into the middle ear. Thereafter, the electrode array proceeds either to the round window or to the location where the cochleastomy will be performed for insertion into the scala tympani of the cochlea. For this embodiment, the implanted module is positioned to extend into the interior of the ear canal. Alternatively, the implanted module is positioned within the soft tissue behind the pinna without entering the ear canal. Instead, the electrode array extends from the implanted module through the soft tissue behind the pinna into the ear canal. Thereafter, the array extends in similar manner through the ear canal, and through or underneath the tympanic membrane, to the middle ear and the nerves of the cochlea.

In alternative and preferred embodiments of the invention, the electrode array is surgically routed from the implanted module in the soft tissue to the cochlea without entering the ear canal. This method of surgical implantation eliminates percutaneous perforation of the ear canal and tympanic membrane, and thereby reduces the chance of infection or damage to that patient. To accomplish these advantages, the electrode array is positioned between the mastoid bone and the skin of the external auditory canal without projecting into the auditory canal. The electrode array is then routed around the tympanic membrane and through the middle ear to the cochlea.

To provide additional support for the array, various modifications can be made to the positioning and routing of the electrode array. For example, a small canal, such as 1-10 mil deep, may be surgically formed in the mastoid bone for receipt of the electrode array as it continues from the implanted module to the inner ear. From there, the electrode array continues to the cochlea in similar manner to that described above. In still an additional preferred embodiment of the invention, a hole is formed in the Spine of Henle, also referred to as the spina suprameatica or Henle's Spine, which is a ridge which projects downward from the mastoid bone adjacent and transverse to the auditory ear canal. The electrode array passes through the hole of the Spine of Henle which prevents the electrode array from being displaced from the side of the mastoid bone and into the auditory ear canal.

A primary object of the present invention is to provide a simple in-the-ear cochlear implant that will overcome the shortcomings of the prior art devices.

Another object is to provide a simple in-the-ear cochlear implant that does not require drilling through the mastoid bone.

Another object is to provide a simple in-the-ear cochlear implant that utilizes a simple single contact electrode with which to stimulate the remaining basilar membrane dendrites or spiral ganglia nerve cells.

Another object is to provide a simple in-the-ear cochlear implant procedure that can be performed with minimal invasive surgery.

Another object is to provide a simple in-the-ear cochlear implant that provides lower costs and less trauma to the patient.

These and other specific objects and advantages of the invention will be apparent to those skilled in the art from a review of the following detailed description taken in conjunction with the drawings.

### BRIEF DESCRIPTION OF THE DRAWINGS

FIG. 1 is a cutaway view of the human ear anatomy with the cochlear implant of the present invention;

FIG. 2 is a cutaway view of the human ear anatomy illustrating a second embodiment of the cochlear implant of the present invention;

FIG. 3 is a cutaway view of the human ear anatomy illustrating a third embodiment of the cochlear ear implant of the present invention including a behind-the-ear module;

FIG. 4 is a cutaway view of the human ear anatomy illustrating a fourth embodiment of the cochlear ear implant positioned below the ear canal;

FIG. 5 is a cutaway view of the human ear anatomy illustrating a fifth embodiment of the cochlear ear implant of the present invention including an in-the-ear module;

FIG. 6 is a cutaway view of the human ear anatomy illustrating a sixth embodiment of the cochlear ear implant of the present invention;

FIG. 7 is a cutaway view of the human ear anatomy illustrating a seventh embodiment of the cochlear ear implant of the present invention including an electrode array that passes through the tympanic membrane;

FIG. 8 is a cutaway view of the human ear anatomy illustrating a eighth embodiment of the cochlear ear implant of the present invention including an behind-the-ear module;

FIG. 9 is a cutaway view of the human ear anatomy illustrating a ninth embodiment of the cochlear ear implant of the present invention including an electrode that passes through the mastoid bone;

FIG. 10 is a cutaway view of the human ear anatomy illustrating a tenth embodiment of the cochlear ear implant of the present invention including a behind-the-ear module a speaker assembly positioned in the ear canal and an electrode that passes through the tympanic membrane;

FIG. 11 is a cutaway view of the human ear anatomy illustrating an eleventh embodiment of the cochlear ear implant of the present invention including a behind-the-ear module and electronic components positioned in the soft tissue behind the pinna and in the mastoid bone;

FIG. 12 is a cutaway view of the human ear anatomy illustrating a twelfth embodiment of the cochlear ear implant of the present invention which is a hybrid of cochlear implant and hearing aid technologies;

FIG. 13A and FIG. 13B are side views an exterior behind-the-ear module and an interior module implanted in the soft tissue behind the pinna illustrating a hardwired connection between the two; and

FIG. 14A, FIG. 14B and FIG. 14C are side views an exterior behind-the-ear module and an interior module implanted in the soft tissue behind the pinna illustrating an electromagnetic connection between the two.

### DETAILED DESCRIPTION OF THE INVENTION

While the present invention is susceptible to the embodiment in various forms, as shown in the drawings, hereinafter will be described the presently preferred embodiments of the invention with the understanding that the present disclosure is to be considered as a exemplification of the invention and is not intended to limit the invention to the specific embodiments illustrated.

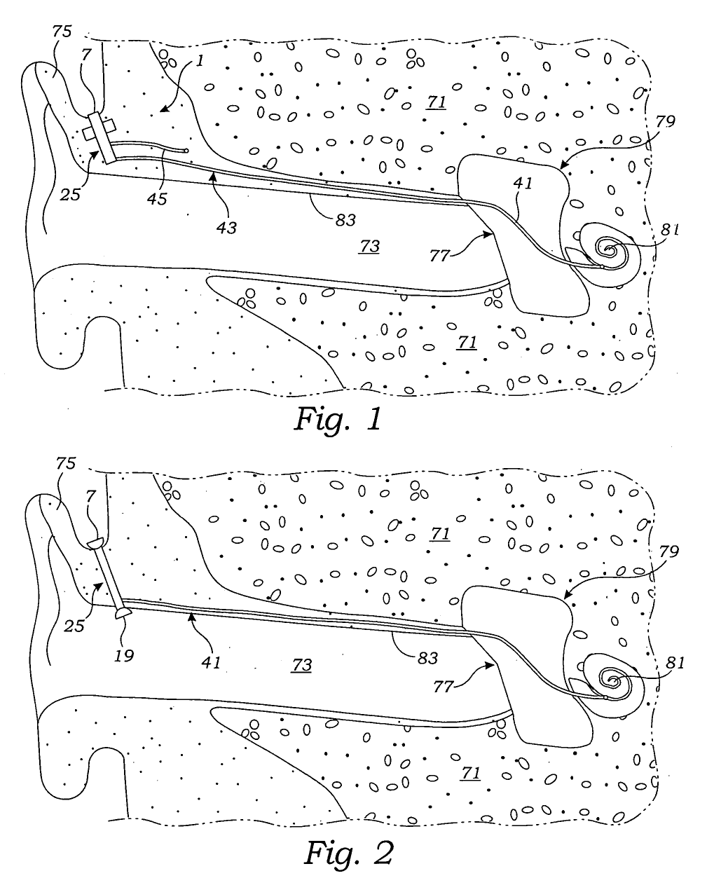

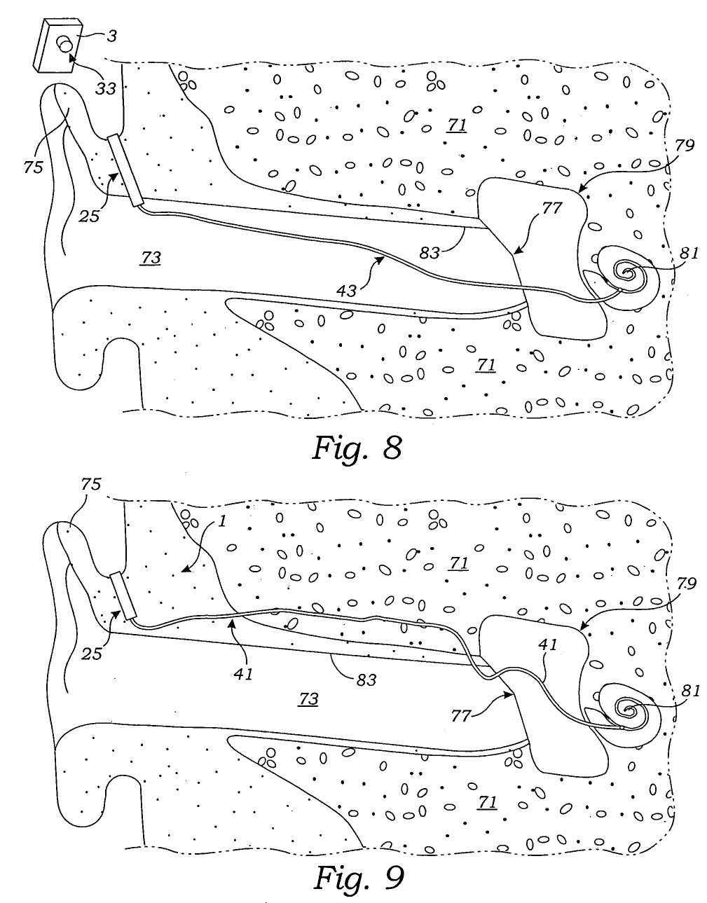

With reference to FIGS. 1-12, the cochlear implant 1 of the present invention includes one or more modules for housing a power supply and the active and passive electronics for processing sound into electrical stimuli which can be interpreted by the brain as sound. In addition, the cochlear implant 1 includes an electrode array 41 for stimulating the nerves of the cochlea.

As shown in FIGS. 1, 2, 4, 6, 7, and 9, the cochlear implant may include a single modular unit 25 which houses all of the active electronics including processor, power supply and microphone and is positioned within the soft tissue behind the ear pinna 75. In operation, the active electronics perform all steps in receiving and conditioning sound waves for the creation and transmission of stimulus signals to the cochlear electrode assembly. The conditioning steps may include amplification, filtering, and conversion into stimulus signals which are interpreted by the brain as sound. As will be explained in greater detail below, the stimulus signals may be altered through transmission through the various components. For example, the processing may utilize either pulse position or pulse width modulations to stimulate the hearing nerve of the cochlea.

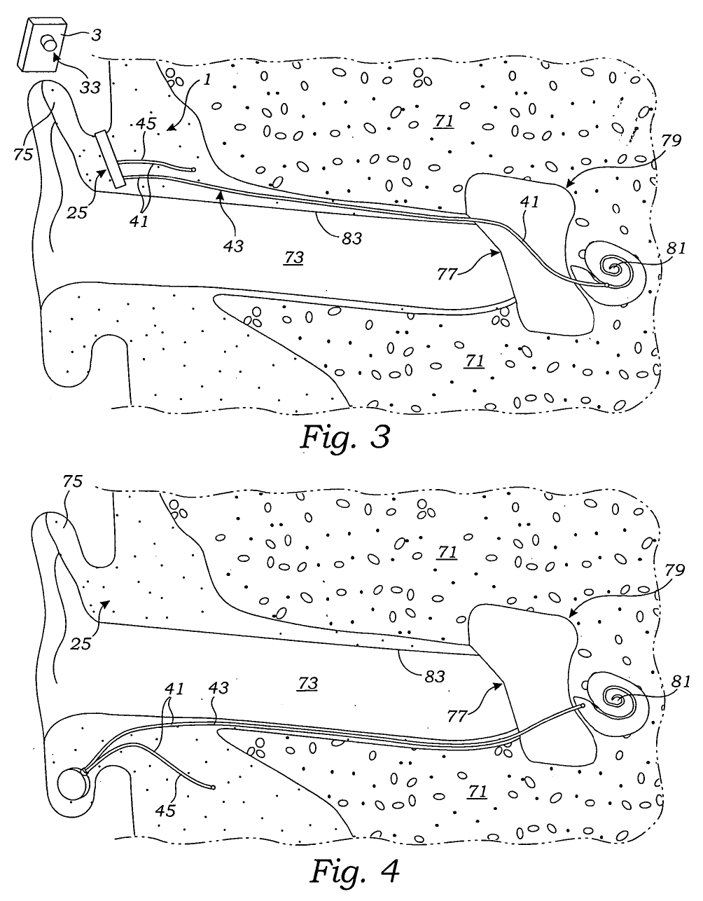

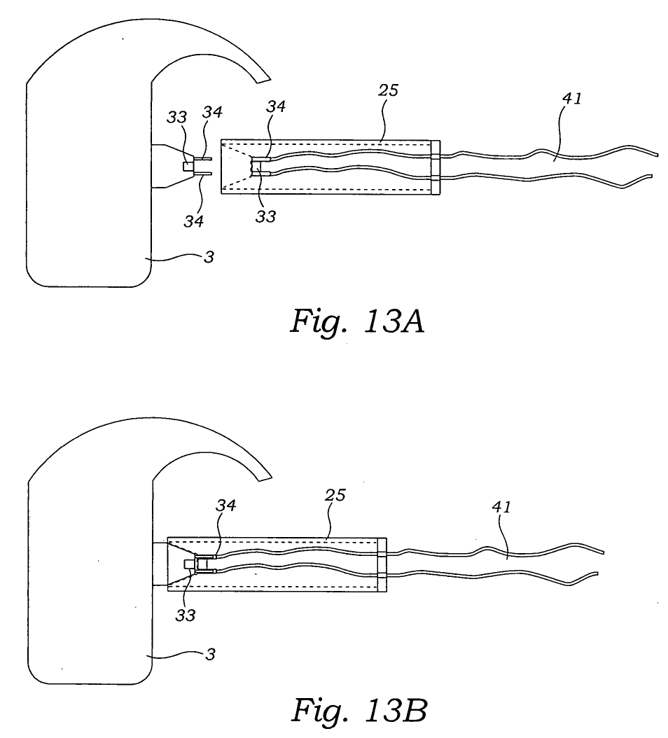

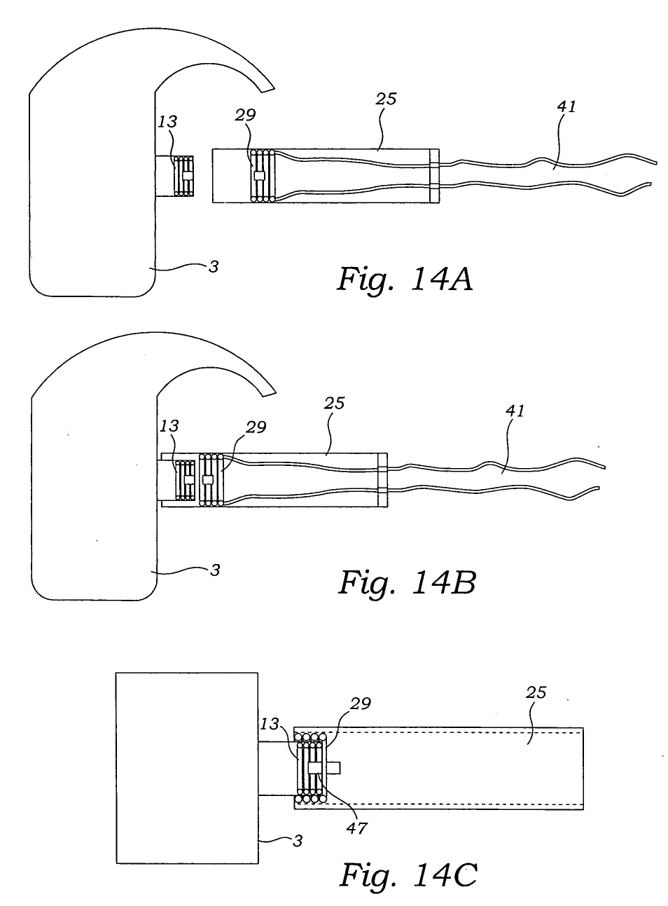

In the alternative to including only a single module, as shown in FIGS. 3, 5, 8 and 10-12, the cochlear implant 1 has two modular units including an interior module 25 and an exterior module 3. The implanted module 25 is implanted within the soft tissue between the ear pinna and mastoid bone. Meanwhile, the second unit 3 is an external module which may be positioned at various external locations such as behind-the-ear (BTE) or within the ear canal. Still with reference to FIGS. 3, 5, 8 and 10-12, the exterior ear module 3 is removable, and is preferably constructed in the same manner as that of a conventional Behind-The-Ear (BTE) as shown in FIGS. 13 and 14. The exterior module 3 includes a microphone 7 that senses acoustic pressure waves and then converts them to electrical signals. The electrical signals are processed by a signal processor 9, which typically provides amplification and conversion into signals designed to stimulate nerves within the cochlea.

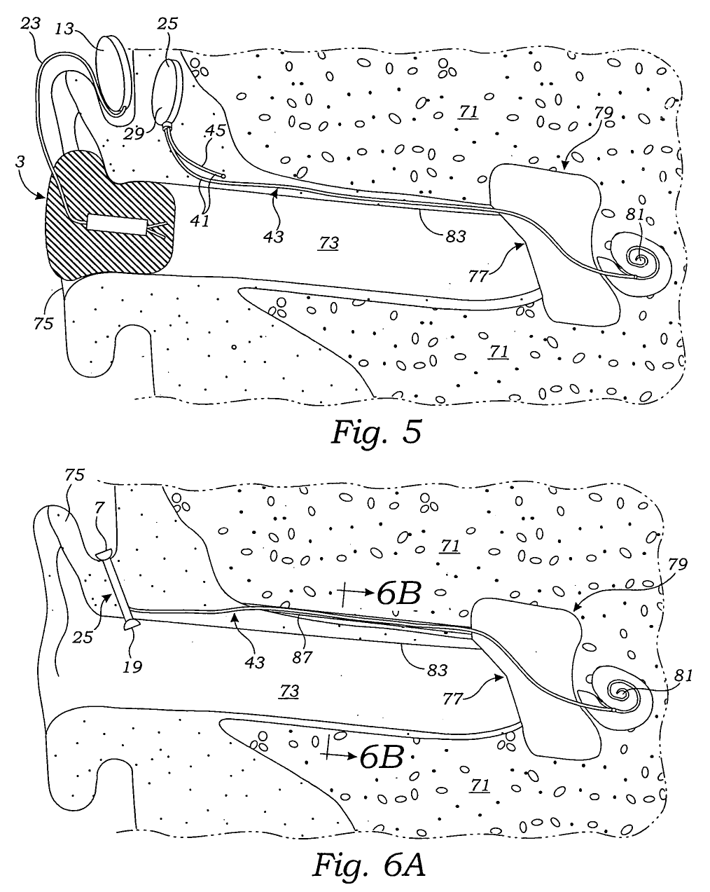

As shown in FIGS. 5, 10 and 12, the exterior ear module 3 may include a behind-the-ear unit and an in-the-ear unit. This construction is considered preferable where patients have extremely small auditory canals, or where a patient is not particularly concerned with the aesthetics of a behind-the-ear module which is more visible to the public. For this embodiment, the microphone, processor and power supply are preferably located within the behind-the-ear unit. The cochlear implant includes a wire 23 for transmitting auditory to the in-the-ear unit. As would be understood by those skilled in the art, the exterior ear module 3 and behind-the-ear module 37 may be constructed in various forms. For example, as shown in FIGS. 13 and 14, preferably the behind-the-ear module 37 includes an ear hook arm 39 for assisting the module in residing upon a patient's pinna. Moreover, though not shown in the Figures, in-the-ear unit may, or may not, include ventilation vents which extend along its length to allow ventilation throughout the patient's auditory canal.

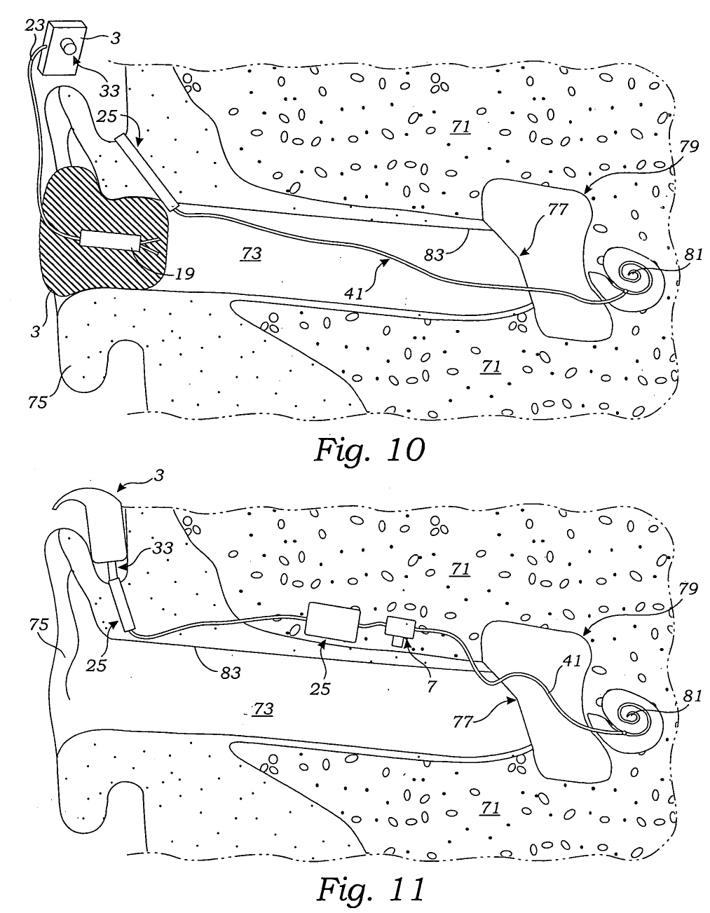

With reference to FIGS. 3 and 10, where the cochlear ear implant 1 of the present invention includes an exterior module 3, the cochlear implant also includes a passive interior ear module 25. As opposed to the easily removable exterior ear module, the implanted ear module is surgically implanted in the soft tissue behind the pinna. Preferably, the implanted module is provided in the shape of an elongate tube having a sufficiently small diameter so that it can be inserted into the soft tissue between the pinna and mastoid bone by a “piercing” operation. The implanted ear module is of simple construction and preferably does not contain active electronics. Instead, it is preferably a simple passive module for relaying signals to the electrode array. The purpose of the implanted ear module is to receive signals, which may be pulsatile or amplitude modulated in nature, from the exterior ear module and convey those signals to the cochlear electrode array.

If the cochlear implant of the present invention includes an exterior ear module 3, preferably, it is selectively connectable and disconnectable to the interior ear module. The selective electrical coupling between modules can be accomplished by various means. For example, as shown in FIG. 13, the modules may be electrically connected using simple quick connect/disconnect connectors 33 having a plurality of electrical contacts 34. If transmitting signals using an electrical connector, the connector should be biocompatible and miniature in construction and provide relative ease of connectability and accessibility for a surgeon. Alternatively, as shown in FIG. 14, it is believed that the preferred manner for communicating auditory signals from the exterior ear module to the interior ear module is accomplished by an electromagnetic induction link which does not require physical contact between components.

Instead, as shown in the FIG. 14, the exterior ear module 3 is constructed to include a circular primary coil 13 concentrically aligned with a central axis. Meanwhile, the interior ear module 25 includes a secondary coil 29 that produces a variable electric current in response to the variable electromagnetic field produced by electrical signals sent to the primary coil 13. Preferably, the implanted module 25 also includes a central cavity. The use of the central cavity aids in axial alignment between ear modules 3 and 25. Alternatively, the inductive coupling may be constructed in a reverse manner in which the secondary coil 29 of the implanted module 25 is sized and positioned to project into a cavity formed within the exterior module 3. Still additional inductive coil constructions can be devised by those skilled in the art.

Preferably, the primary induction coil is a flat wound shaped coil. The primary coil 13 transmits the electrical signals to secondary coil through an electromagnetic coupling. The secondary coil 29 integrates this induction signal and passively extracts audio information transmitted by the primary coil to produce stimulus signals. Across this electromagnetic link, audio information is transmitted as well as energy to power the electronics, if any, of the implanted module 25. This method of coupling removes any angular coil coupling issues and greatly diminishes the coupling losses due to coil separation. The inductive coil construction also results in a very efficient energy transfer from the exterior module to implanted module. The efficiency of this inductive coupling is strongly influenced by the proximity of the two coils. Thus, the coils are desirably placed as close to each other as possible. Moreover, algorithms may implemented within the exterior or implanted module that allow for various methodologies of electrode stimulation.

The cochlear ear implant of the present invention also includes an electrode array 41. The electrode array is preferably a simple structure including the implanted active electrode 43, the return electrode 45, and a biocompatible miniature connector (not shown) which connects the active and return electrodes to the interior ear module. A specially manufactured connector is necessary due to the physically small size required and the need for biocompatibility. The electrode array preferably includes an electrode have a single strand of wire having a diameter of 5-10 mil (one thousandth of an inch) coated with an insulator 5-10 microns (one millionth of an inch) thick. Preferably, the electrode wire is made of particularly soft metal such as substantially pure platinum. Alternatively, the electrode wire includes an uncharacteristically high amount platinum to iridium having a ratio of greater than 90%:10%. Meanwhile, the wire strands are preferably coated with an insulation of Parylene.

The electrode array 41 extends from the implanted module of either the single module construction or double module construction by various paths to the cochlea where the distal end of active electrode 43 is implanted within the scala tympani duct. The distal extremity of the implanted electrode is typically inserted into the scala tympani of the cochlea 81. The return electrode is typically located extracochlearly in the middle ear cavity. The return electrode may be constructed as a small conductive mesh region to enable a low impedance tissue connection and is also silver or platinum. This placement and construction of the active and return electrodes facilitates current spreading and the resultant stimulation of a larger population of neurons. Possible structural variations of the cochlear electrode include manipulating the active electrode shape and orientation to better project the stimulating current towards the modiolus region. Preferably, the terminus of the active electrode is approximately 6 mm, much shorter than that of traditional multielectrode arrays. The shortness of this electrode significantly reduces trauma to the cochlea, minimizing the chances of compromising a patient's residual hearing. The patient is then able to pursue future hearing technological developments.

In preferred embodiments, implantation of the electrode array does not require surgical excavation of the mastoid bone to route the electrode array from the implanted module to the cochlea. Instead, the electrode array is positioned to pass between the ear canal skin 83 and mastoid bone 71, and through or underneath the tympanic membrane into the middle ear 79. Thereafter, the electrode array proceeds either to the round window or to the location where the cochleastomy will be performed for insertion into the scala tympani of the cochlea.

The medical procedure for surgical implantation of the cochlear implant may be accomplished using various methodologies using a variety of cochlear implant constructions. For example, with reference to FIG. 1, a preferred cochlear implant 1 of the present invention, includes a single housing 25 implanted within the soft tissue behind the pinna 7. The implanted module has an elongate tube shape which can be inserted into the soft tissue between the pinna and mastoid bone by a “piercing” operation. All active and passive electronics, including processor, power supply and microphone 7 are located in the implanted module 2. The proximal extremity of the module 25 includes a microphone 7 which is positioned just under the patient's skin, or as shown in FIG. 1, the microphone projects exterior of the patient's skin for better acoustic response. Meanwhile, the electrode array is positioned with the return electrode positioned in the soft tissue immediately adjacent to the implanted module 25 and the active electrode 43 is positioned between the mastoid bone 71 and the skin 83 of the external auditory canal 73 without projecting into the auditory canal. The active electrode 43 is then routed around the tympanic membrane 77 through the middle ear 79 to the cochlea 81. Preferably, the distal extremity of the cochlear electrode 43 is inserted into the cochlea in a manner to better project the stimulating current towards the modiolus region.

Cochleastomy Procedure

The above implantation of the cochlear implant can be accomplished through the following procedures. A postauricular incision is made about 3 mm behind the crease. The pinna 75 is then reflected anteriorly, creating an avascular plane up to the posterior bony canal. The posterior canal skin 83 is then elevated down to the annulus of the tympanic membrane 77. A modified Wietlander is inserted to retract the canal skin, exposing the edge of the medial end of the bony canal. With magnification now necessary, the annulus is separated from the bony canal using a Rosen needle and drum elevator. The chordi tympani nerve should be visualized and preserved. The canal skin, in concert with the eardrum 77, is elevated anteriorly up to the malleus, exposing the contents of the middle ear 79, and particularly the promontory and round window.

If more exposure is necessary, some posterior canal bone 71 can be removed with a drill or curret, being cognizant of the facial nerve within close proximity. Attention is then turned to create a pocket under the temporalis facia and muscle and with a Freer elevator. The internal receiver is then placed into the pocket, although one may reserve this until after the electrode is placed into the cochleostomy for easier electrode insertion.

With the internal receiver of the implant properly seeded, one then can create a cochleastomy by drilling just anterior to the round window, following the curve of the promontory which constitutes the basal turn of the cochlea. The depth of the cochleastomy varies but usually one encounters perilymph 3 to 5 mm into the cochlea. With this accomplished the active electrode can be inserted into the cochlea at its full length of 6 mm. The remaining wire is then placed along the bony canal wall. The retractor is removed, covering the wire, and the wound is closed in two layers.

Another modification is indicated if exposure of the medial end of the canal is difficult. This may occur in narrow canals. In this case the posterior canal flap can be split, retracting the lateral end with the retractor and penrose drain. This can be easily replaced at the end of the case, but some packing may be necessary to secure the flaps in place. Also, there would be some retraction at the incision area between the lateral and medial ends of the canal skin possibly exposing the wire. The temporalis fascia can be harvested to cover the electrode at the junction area before the flaps are returned to their anatomical positions. The implanted module is connected to the electrode array and positioned in the soft tissue behind the pinna. The incisions are then closed in a layered fashion.

Modifications

Modifications to the implanted module can be made to its construction and placement without departing from the spirit and scope of the invention. For example, as shown in FIG. 4, the implanted module 25 may be disk shaped or spherically shaped. Moreover, the module 25 may be implanted within the pinna, as opposed to within the soft tissue adjacent the mastoid bone. Further, the implanted module may be positioned above, below or adjacent to the ear canal, in which case the active electrode 43 will preferably be routed in like manner between the mastoid and the ear canal skin 83. Advantageously, this location is easily accessible by a surgeon and is relatively robust in terms of infections. This positioning of the electrode array also provides a reasonably straight access route to the cochlea.

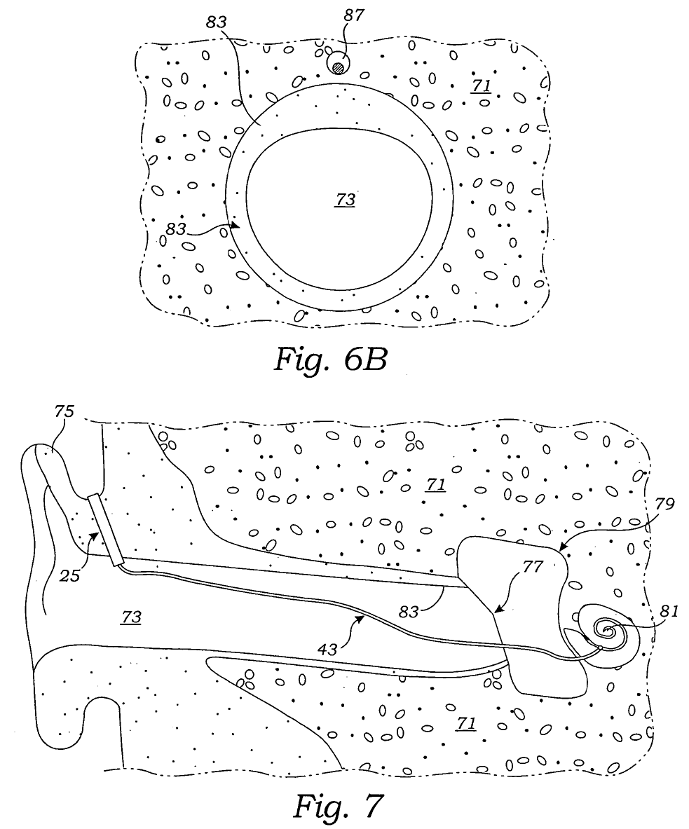

Still additional modifications can be made to the positioning and routing of the electrode array. For example, as shown in FIGS. 6 and 9, a channel 87, preferably 1-10 mil deep, may be surgically formed in the mastoid bone 71 for receipt of the electrode array 41 as it continues from the implanted module to the inner ear. The ear canal skin 83 overlays the mastoid bone 71 to maintain the electrode 41 in place. From between the mastoid bone and ear canal skin 83, the active electrode continues to the cochlea in similar manner to that described above.

In still an additional preferred embodiment of the invention, a hole is formed in the Spine of Henle (not shown), also referred to as the spina suprameatica or Henle's Spine. The Spine of Henle is a ridge which projects downward from the mastoid bone 71 adjacent and transverse to the auditory ear canal. The surgically formed hole is preferably the same size, or slightly larger, than the diameter of the active electrode 43. The active electrode 43 is then surgically inserted through the hole formed in the Spine of Henle and routed between the mastoid bone 71 and the ear canal skin 83. Thereafter, the active electrode passes around the tympanic membrane and through the middle ear 79.to the cochlea 81 in similar manner to that described above. Affixing the electrode array to the mastoid bone by employing the anatomical structure of the Spine of Henle prevents the electrode array from being displaced from the side of the mastoid bone and into the auditory ear canal. Post operatively, the skin of the ear canal will heal, adhering to the mastoid bone, and maintaining the electrode array in place.

In still additional embodiments of the invention, as shown in FIGS. 7, 8 and 10, instead of routing the active electrode 43 between the mastoid bone 71 and the skin 83 of the auditory canal, the active electrode projects into the auditory canal. This embodiment is not considered preferred. However, this procedure and practice may be desirable where a patient has particularly thin skin in the ear canal. The implanted module 25 is positioned in the soft tissue behind the pinna 75. The implanted module 25 is constructed and positioned to project into the interior of the ear canal 73. Alternatively, in an embodiment not shown in the Figures, the implanted module is positioned within the soft tissue behind the pinna without entering the ear canal. Instead, the electrode array extends from the implanted module through the auditory canal skin 83 into the ear canal. With reference again to FIGS. 7, 8, and 10, the array then extends through the ear canal, through the tympanic membrane, to the middle ear and the nerves of the cochlea. To position the electrode array 41, an incision is made through the tympanic membrane 77 and the active electrode 43 is manually forced through the incision. Thereafter, it is preferred that the active electrode 43 is positioned to engage the cochlea's nerve cells. Over the next days and weeks, the tympanic membrane heals around the electrode array 41 thereby providing a substantially gaseous seal.

In still an additional embodiment, as shown in FIG. 11, minor surgical excavation of the mastoid bone is conducted for placement of a microphone 7 and the implanted module 25 which contains the necessary audio processor, modulator and preamplifier. This procedure and cochlear implant construction is not considered preferred, as it is preferred that excavation of the mastoid bone be completely avoided. However, implantation of the microphone within the mastoid bone interior to the ear canal 73 makes use of the ear's natural acoustics, providing a more natural sound to the user. In addition, the placement of the power supply in an exterior module 3 maximizes power capacity.

As shown in the Figures, still more modifications can be made to the cochlear ear implant of the present invention. For example, recently it has been understood that low frequency acoustic energy to the tympanic membrane can assist those with hearing in the lower portion of the audio spectrum by providing both electrical and acoustic stimuli. More particularly, it has recently been determined that a significant percentage of cochlear implant candidates retain usable residual hearing in the lower frequency ranges of the audio spectrum. By providing both electrical and acoustic stimuli, significant gains can be obtained by the patient. Accordingly, where the patient has residual hearing, the cochlear implant 1 may be constructed to include a speaker assembly 19. For example, as shown in FIGS. 2 and 6, the implant module 25 incorporates a speaker assembly 19 which projects from the soft tissue behind the pinna 75 through the canal wall 83 into the ear canal 73. In the alternative, as shown in FIG. 10, the cochlear implant may include an exterior module 3 incorporating a speaker assembly 19 located in the ear canal. Where the cochlear ear implant includes a speaker, preferably the speaker includes audio filters for producing sound only in the lower frequency ranges, while the electrical stimulation through the electrode array is filtered to produce only higher frequency stimuli. Preferably, a mutual crossover frequency is established between the acoustic signal spectrum and electrical spectrum. Also, caution must be exercised so as to avoid acoustic feedback.

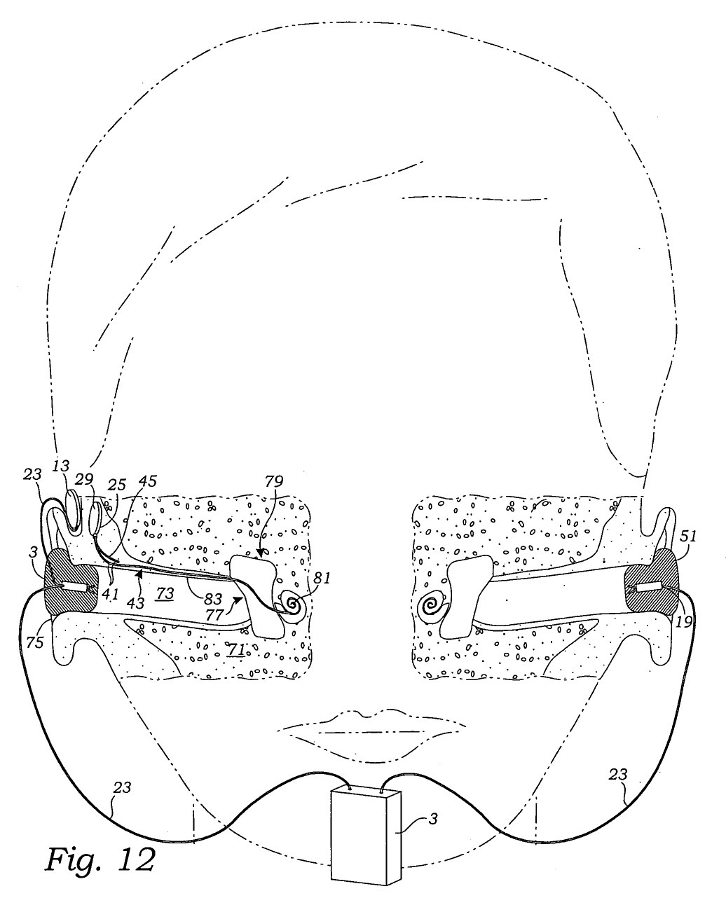

As shown in FIG. 12, the cochlear implant of the present invention may include a hearing aid component 51 in the non-implanted ear. The cochlear implant includes an exterior processing unit 3 which stores the active and passive electronics including microphone, battery and processor. The exterior unit is preferably connected to an implant unit through a transcutanteous induction link employing a primary coil 13 and a secondary coil 29 to transmit signals through the electrode array to the cochlea. Again, it is preferred that the electrode array 41 be positioned between the mastoid bone 71 and the auditory canal skin 83. As shown, the cochlear implant may incorporate a speaker assembly 19 located in the ear canal 73 in the event the patient has residual hearing. In addition to the cochlear implant component, a hearing aid component 51 may be provided. The hearing aid is connected to the exterior module 3 with wires 23 to transmit audio signals from the processor to the non-implanted ear. For those patients that have residual hearing in their non-implanted ear, this configuration can save the user from manipulating or purchasing two separate units. Also, this configuration makes possible, performance features such as scaling both ears together. In other words, if the user needs to change the volume control on one unit, the other will be scaled accordingly. Also, providing the single processor for acoustic response to both ears makes it easier to match the phasing of the signals sent to the ears, enabling superior localization. Directional microphones (such as sold by Knowles Electronics or Sonion) can also be used to provide a directional response. With the addition of directional microphones, a directional pattern is established for the patient. Moreover, spatial filtering is employed which provides an Al-DI (Articulation Index weighted Directivity Index) of 5 dB or better to provide an immediate and noticeable improvement in typical real world listening situations.

In operation, the microphone(s) of the cochlear implant convert(s) the ambient sound environment into an electrical analogy. A state of the art digital signal processor (DSP) based audio processor creates the stimulus signals through necessary signal processing and conditioning using amplification, compression, expansion, threshold adjustments and noise canceling algorithms. Most modern DSP processors utilize either pulse position or pulse width modulation methods of outputting a signal, which typically would be routed to the speaker for conversion to acoustic energy. Instead, preferably the output of the audio processor is a pulsatile waveform that is modulated with audio information to power the primary coil of the induction coupling system. The interior ear module, which contains the secondary coil, then integrates this waveform and extracts the embedded audio information. The audio information is then transmitted through the interior ear module to the cochlear electrode array to stimulate the hearing nerves. This nerve stimulation is then interpreted by the brain as sound.

To power this cochlear implant, a conventional hearing aid battery can be used, or a rechargeable lithium based, or other chemistry battery. The recharging of this battery can be accomplished using induction methods in which the primary coil, located in the exterior ear module, is configured to receive the induction recharge energy. This feature greatly adds to the user's convenience and ease of operation.

Various modifications of the cochlear implant may be made. For example, a possible functional variation involves the usage of two microphones, instead of one, that provides a cardioid like spatial response pattern to the user. Any improvement in signal-to-noise (S/N) ratio, that results from using directivity, is of major importance to the hearing impaired. By using two microphones configured to provide a cardioid response, the desirable increase in S/N ratio is achieved through spatial filtering. An extension of this would be to provide a pair of microphones, per ear, that would communicate with each other via a low power, miniature RF or inductive link, thus creating a four microphone array. The directionality of such an array would provide for even greater spatial filtering resulting in a further increase in S/N that is so critical for the hearing impaired. A variation of this approach would be to provide for a radio receiver within the exterior ear module that would receive a signal transmitted by a desk-top or handheld directional microphone array. This type of array would provide additional directionality improvement and provide further capability in attenuating unwanted environmental noises.

Still additional modifications of the cochlear ear implant can be made. For example, the invention has been described predominantly using digital signal process to produce pulsatile waveforms that are modulated with audio information to power the primary coil of the induction coupling system. However, the cochlear ear implant of the present invention is also capable of using amplitude modulation methods. For example, the audio input produced by the microphone can be modulated by an amplitude modulator to produce an amplitude modulated electromagnetic field from the primary coil. Meanwhile, the interior ear module includes a passive amplitude demodulator for converting the electromagnetic waves into stimulus signals recognizable by the brain through the cochlear nerves.

While several particular forms of the invention have been illustrated and described, it will be apparent that various modifications can be made without departing from the spirit and scope of the invention. Accordingly, it is not intended that the invention be limited except by the following claims.

I claim:

1. A cochlear implant for an ear having an external auditory canal, a: middle ear, a cochlea, a tympanic membrane, and a scala tympanic, the cochlear implant comprising:
a microphone positioned exterior to the tympanic membrane for converting sound waves into microphone electrical signals;
a processor for converting said microphone electrical signals to stimulus signals adapted to stimulate nerves within the cochlea;
a power source for said microphone and processor;
an implantable soft tissue module connected to said microphone for transmitting signals from said microphone and processor to an electrode array, said soft tissue module located at least partially within the soft tissue between the pinna and mastoid bone; and
an implantable electrode array extending from said soft tissue module to said cochlea for transmitting said stimulus signals from said processor to stimulate nerves within the cochlea, said electrode array positioned between the skin of the external auditory canal and the mastoid bone and routed around the tympanic membrane into and through the middle ear to the cochlea.

2. The cochlear implant of claim 1 wherein said electrode array is positioned within a channel formed surgically into the mastoid bone below the skin of external auditory canal.

3. The cochlear implant of claim 1 wherein said soft tissue module is positioned, at least partially, within a recess formed from excavating a portion of the mastoid bone behind the pinna without forming a hole through the mastoid bone.

4. The cochlear implant of claim 1 wherein said electrode array is routed through a hole formed through the Spine of Henle.

5. The cochlear implant of claim 1 wherein said microphone is located within said soft tissue module.

6. The cochlear implant of claim 1 wherein said module is tubular shaped.

7. The cochlear implant of claim 1 wherein said electrode is a single strand wire having a diameter of 5-10 thousandth of an inch coated with an insulator 5-10 microns thick.

8. The cochlear implant of claim 1 wherein said microphone and processor are located in said implantable soft tissue module.

9. The cochlear implant of claim 1 further comprising:
a removable exterior ear module including said microphone, said processor and said power source, said exterior ear module positioned within the exterior portion of said auditory canal and removable from said auditory canal without surgery; and
said soft tissue ear module connectable and disconnectable to said exterior ear module, said implantable ear module electrically connecting said exterior ear module to said electrode array for relaying stimulus signals from said processor to said electrode array.

10. The cochlear implant of claim 9 wherein:
said exterior ear module includes a primary coil for converting signals produced by said processor into electromagnetic signals; and
said implantable ear module includes a secondary coil for converting said
electromagnetic signals into stimulus signals.

11. The cochlear implant of claim 1 further comprising a speaker assembly producing acoustic signals.

12. The cochlear implant of claim 1 further comprising a speaker assembly positioned within the ear canal.

13. A cochlear implant for an ear having an auditory canal, a middle ear, a cochlea, a tympanic membrane, and a scala tympanic, the cochlear implant comprising:
a microphone positioned exterior to the tympanic membrane for converting sound waves into microphone electrical signals;
a processor for converting said microphone electrical signals to stimulus signals adapted to stimulate nerves within the cochlea;
a power source for said microphone and processor;
an implantable soft tissue module connected to said microphone for relaying signals from said microphone and processor to an electrode array, said soft tissue module positioned within a recess formed from excavating a portion of the mastoid bone behind the pinna without forming a hole through the mastoid bone; and
an implantable electrode array extending from said soft tissue module to said cochlea for transmitting said stimulus signals from said processor to stimulate nerves within the cochlea, said electrode array positioned between the skin of the auditory canal and the mastoid bone and through a hole formed in the“Spine of Henle”, said electrode array also passing around the tympanic membrane and projecting into and through the middle ear to the cochlea.

14. The cochlear implant of claim 13 wherein said electrode array is positioned within a channel formed surgically into the mastoid bone below the skin of external auditory canal.

15. The cochlear implant of claim 13 wherein said microphone is located within said soft tissue module.

16. The cochlear implant of claim 13 wherein said module is tubular shaped.

17. The cochlear implant of claim 13 wherein said electrode is a single strand wire having a diameter of 5-10 thousandth of an inch coated with an insulator 5-10 microns thick.

18. The cochlear implant of claim 13 wherein said microphone and processor are located in said implantable soft tissue module.

19. The cochlear implant of claim 13 further comprising:
a removable exterior ear module including said microphone, said processor and said power source, said exterior ear module positioned within the exterior portion of said auditory canal and removable from said auditory canal without surgery; and
said soft tissue ear module connectable and disconnectable to said exterior ear module, said implantable ear module electrically connecting said exterior ear module to said electrode array for relaying stimulus signals from said processor to said electrode array.

20. The cochlear implant of claim 19 wherein:
said exterior ear module includes a primary coil for converting signals produced by said processor into electromagnetic signals; and
said implantable ear module includes a secondary coil for converting said
electromagnetic signals into stimulus signals.

21. The cochlear implant of claim 13 further comprising a speaker assembly producing acoustic signals.

22. The cochlear implant of claim 13 further comprising a speaker assembly positioned within the ear canal.

23. A method of implanting a cochlear implant in a patient comprising the steps of:
providing a cochlear implant system including a microphone for converting sound waves into microphone electrical signals, a processor for converting said microphone electrical signals to stimulus signals adapted to stimulate nerves within the cochlea, a power source for said microphone and processor; an implantable electrode array for transmitting said stimulus signals. from said processor to stimulate nerves within the cochlea and an implantable soft tissue module for transmitting signals from said microphone and processor to an electrode array;
providing an incision into the soft tissue behind the ear of a human subject;
elevating the skin of the auditory canal to the annulus of the tympanic membrane;
performing a cochleastomy by forming a hole into the cochlea;
positioning the soft tissue module at least partially within the soft tissue between the pinna and mastoid bone;
directing the electrode array between the skin of the external auditory canal and the mastoid bone and passing it around the tympanic membrane into and through the middle ear to engage the patient's cochlea.

24. The method of implanting a cochlear implant in a patient of claim 23 further comprising the steps of:
forming a hole in the Spine of Henle; and
passing the electrode array through the hole in the Spine of Henle.

25. The method of implanting a cochlear implant in a patient of claim 23 further comprising the step of:
forming a channel into the mastoid bone below the skin of external auditory canal for receipt of the electrode array.

26. The cochlear implant of claim 23 further comprising the step of:
excavating a portion of the mastoid bone behind the pinna without forming a hole through the mastoid bone to provide a recess for at least partial receipt of the implantable soft tissue module.

27. The method of implanting a cochlear implant in a patient of claim 23 further comprising the step of positioning the microphone, processor and power supply in the soft tissue module.

28. The method of implanting a cochlear implant in a patient of claim 23 further comprising the step of positioning the microphone, processor and power supply in a behind-the-ear module located behind the patient's pinna.

29. A cochlear implant for an ear having an external auditory canal, a middle ear, a cochlea, a tympanic membrane, and a scala tympanic, the cochlear implant comprising:
a microphone positioned exterior to the tympanic membrane for converting sound waves into microphone electrical signals;
a processor for converting said microphone electrical signals to stimulus signals adapted to stimulate nerves within the cochlea;
a power source for said microphone and processor;
an implantable module connected to said microphone for transmitting signals from said microphone and processor to an electrode array; and
an implantable electrode array extending from said implantable module to said cochlea for transmitting said stimulus signals from said processor to stimulate nerves within the cochlea, said electrode array including an active electrode and a return electrode, said active electrode constructed of a single strand wire having a diameter of 5-10 mil coated with an insulator 5-10 microns thick

30. A cochlear implant of claim 29 wherein said active electrode has greater than 90% of platinum.
